(19) United States
(12) Patent Application Publication
(10) Pub. No.: US 2024/0185986 A1
(43) Pub. Date: Jun. 6, 2024

(54) Method and System for Determining the Effect of Food and Drink on a User

(71) Applicant: My Levels Limited, London (GB)

(72) Inventors: Laura Helen DOUGLAS, London (GB); Iliyan Radev ZAROV, London (GB)

(21) Appl. No.: 18/556,254

(22) PCT Filed: Apr. 19, 2022
(86) PCT No.: PCT/GB2022/050985

§ 371 Date: Oct. 19, 2023

(30) Foreign Application Priority Data
Apr. 19, 2021 (GB) 2105593.4

(51) Int. Cl. G16H 20/60

(52) U.S. Cl. CPC G16H 20/60

(57) ABSTRACT

A method of determining the relative impact of food and drink on the body fluids of a user includes receiving event data of a plurality of events, wherein the events comprise the user consuming food or drink, the event data comprising the type of food or drink and the time at which said event occurred; receiving indicator information over the time corresponding to said events, said indicator information being a numerical measure of a physiological parameter; fitting a mixture of probability density functions to the indicator information, wherein each probability density function represents the probability that an event of the events impacted the indicator information at a time; and extracting a weighting from each fitted probability density function and assigning an impact value based on this weighting for the corresponding event, to allow an impact value to be assigned to an event where the user consumed food or drink.

### FIELD

Embodiments described herein are concerned with determining the effect of food & drink on a user.

### BACKGROUND

Determining how food can affect the blood and/or cellular fluid of a person can help a person to regulate their food and drink intake to help with weight control, control of mood etc.

For example, determining a person's sugar levels helps the person understand their body and what foods they should eat. Spiking sugar levels can make a person put on weight, and crashing sugar levels cause cravings and anger.

However, everyone's body is unique and so everybody's responses are unique. Someone might spike from apples, but not ice cream . . . and another person could be the other way around. If someone experiences a spike in their sugar levels after eating a certain then they are likely to feel far more hungry after this food than they would eating a food which contained the same calories, but did not cause such a large spike.

### BRIEF DESCRIPTION OF THE FIGURES

Embodiments will now be described with reference to the following figures in which:

FIG. 1 is a schematic of a system in accordance with an embodiment;

FIG. 2 is a schematic of a system in accordance with an embodiment shown in more detail to that of FIG. 1;

FIG. 3 is a flowchart showing a method in accordance with an embodiment;

FIG. 4 is a schematic of a user interface for collecting event information from a user where the event information relates to food and drink;

FIG. 5 is a schematic of a user interface for collecting event information from the user where the event information relates to physical activity;

FIG. 6 is a plot of raw data concerning indicator information;

FIG. 7 is a plot of the pre-processed raw data concerning indicator information;

FIG. 8 is a diagram indicating how the raw data is processed;

FIG. 9 is a data plot showing processed indicator information and how this data is fitted using normal distributions;

FIG. 10 is a data plot showing processed indicator information and how this data is fitted using lognormal distributions;

FIG. 11(a) is a schematic plot showing how processed data is analysed and FIG. 11(b) is a plot showing the fitting of the data of FIG. 11(a);

FIG. 12 is a plot showing a fitting of the data;

FIG. 13 is a schematic of a user display showing the ranked food & drink;

FIG. 14 is a schematic of a user display showing the user's indicator information throughout the day and how it has been affected by certain foods; and

FIG. 15 is a schematic of a user display showing the ranked food & drink for a “snack” and a “drink”.

### DETAILED DESCRIPTION OF THE FIGURES

In an embodiment, a method of determining the relative impact of food and drink on the body fluids of a user is provided, the method comprising:
receiving event data of a plurality of events, wherein the events comprise the user consuming food or drink, the event data comprising the type of food or drink and the time at which said event occurred;
receiving indicator information over the time corresponding to said plurality of events, said indicator information being a numerical measure of a physiological parameter;
fitting a mixture of probability density functions to the indicator information, wherein each probability density function represents the probability that an event of the plurality of events impacted the indicator information at a time; and
extracting a weighting from each fitted probability density function and assigning an impact value based on this weighting for the corresponding event, to allow an impact value to be assigned to an event where the user consumed food or drink.

In the above embodiment, the relative impact value indicates which types of food and drink are more likely to create a spike in a user, based on the actual response data of the user. Continuous glucose monitoring permits the recording of glucose levels in a human or animal body over periods with a relatively short interval between measurements.

The above method can be configured to output a warning level for a food or drink, wherein said warning level indicates the rise in the indicator information expected by consuming the food or drink, said warning level being determined from the impact value for said food or drink. The warning levels may be assigned to different bands of impact values.

For example, there may be at least two warning levels, where one of the warning levels indicates food or drink for the user to avoid and the other level indicates food or drink for the user to consider. In an embodiment, the method is configured to display food and drink and drink on a screen indicating the warning level. For example, the name of the food or drink may be assigned a certain colour, e.g. red or green. If more levels are added, then other colours, for example, amber may also be added. However, the warning levels may be outputted in other ways, for example, where the food or drink is displayed, whether it is displayed at all. Also, the impact value may be displayed.

The above allows the output of a ranked list of food and drink using said impact value for ranking.

In a further embodiment, the method is further configured to retrieve from a database an impact value for a food or drink from a plurality of users and provide to a user a comparison of their impact score for a food or drink with the average impact score from the same food of drink determined from the plurality of users. This allows the user to see how they impact value compares with other users for a specific food. This might be helpful for the user to determine underlying health conditions. For example, if a user often has much higher impact values that the average, this might suggest that the user is developing Type II diabetes.

In an embodiment, a mixture model of pdfs comprising a mixture of weighted pdfs where each pdf has a weight applied to it. Although pdfs are used above, in further embodiments, any mixture model could be used comprising a weighted combination of parameterised functions.

In a further embodiment, the above method is adapted to compensate for a user changing their level of physical activity. By modelling the effect of physical activity on the indicator information, it is possible to isolate the effect on the indicator information caused by food alone.

In one embodiment the relative impact of food and drink on the blood sugars of a user, wherein the indicator information is an indication of the blood sugar levels. For example, glucose may be used.

In further embodiments, the indicator information may relate to ketones, insulin, cortisol, oxygen, ghrelin and other hormones—these may be measured on a similar timescale or could also be on a different time scale to the one described herein for glucose, for example, one measurement per day.

The probability distributions may be asymmetric distributions as sugar levels are expected to rise quicker than they fall in response to consuming food and/or drink. However, for events such as physical activity, a symmetric pdf may be more appropriate.

An example of a suitable asymmetric distribution is a log normal distribution.

In a further embodiment, each probability distribution of the mixture of probability distributions is a product of two distributions, wherein the first distribution is configured to model a peak in the indicator information and the second distribution is used to model the decay of the peak. The second distribution may be in the form of an exponential decay.

For example the mixture of pdfs may be written as:

$${P}^{′} ( x ) = {\sum }_{i = 1}^{N} {\prod }_{i} {f}_{i} ( x ) {g}_{i} ( x )$$

Where x is time, i is the event index, πᵢ is a weighting to be fitted, fᵢ(x) is the first distribution and gᵢ(x) is the second distribution.

In an embodiment,

$${f}_{i} ( x ) = \{ \begin{matrix}\frac{1}{( x - {T}_{i} ) {σ}_{i} \sqrt{2 π}} & {e}^{- \frac{{( ln ( x - {T}_{i} ) - {μ}_{i} )}^{2}}{2 {σ}^{2}}} \\ 0 & x \leq {T}_{i}\end{matrix}$$

Where μ is the mean of the variable's natural logarithm and σ is the standard deviation of the variable's natural logarithm and Tis the timeshift of the probability distribution from the origin, and the origin being at x=T₀.

gᵢ(x)=λe^{-λ(x-(T}^{ᵢ}^{+D))}

where

Tᵢ denotes the time at which the food was eaten i.e. the food event was logged by the user, D denotes the time after the food has been logged by the user and the time delay after which the exponential weight decay comes into effect, and λ is a fitting parameter which is the rate parameter of the exponential distribution.

The same λ and D can be used for all users or they may be determined for each user individually. The same λ and D can be used for all food and drink for the same user. In an embodiment, λ and D are set for groups of users sharing the similar characteristics. For example, users with type II diabetes might use the same λ and D whereas non-diabetic users might use a different λ and D.

Fitting the mixture of probability density functions to the indicator information may comprise initialising the probability density functions using the time of the event to be fitted and a value representing the weight of probability density function derived from the event data.

In a further embodiment, a method of determining the relative impact of food and drink on the body fluids of a user is provided, the method comprising:
receiving event data of a plurality of events, wherein the events comprise the user consuming food or drink, the event data comprising the type of food or drink and the time at which said event occurred;
receiving indicator information over the time corresponding to said plurality of events, said indicator information being a numerical measure of a physiological parameter;
fitting a weighted mixture of parameterised mathematical functions to the indicator information, such that each mathematical function of said mixture is provided to model the effect on the indicator information of one event of said plurality of events; and
extracting a weighting from each fitted mathematical function and assigning this weighting as an impact value for the corresponding event, to allow an impact value to be assigned to an event where the user consumed food or drink.

The above method uses a weighted mixture of parameterised mathematical models in the same way that a weighted mixture of pdfs is used in the first embodiment.

In a further embodiment, a method of determining the relative impact of food and drink on the body fluids of a user, the method comprising:
receiving event data of a plurality of events, wherein the events comprise the user consuming food or drink, the event data comprising the type of food or drink and the time at which said event occurred;
receiving indicator information over the time corresponding to said plurality of events, said indicator information being a numerical measure of a physiological parameter;
fitting a plurality of discrete mathematical functions to the indicator information, wherein each mathematical functions represents the relationship between an event and a local maxima, minima or inflection point in the indicator information; and
extracting a weighting from each fitted mathematical function and assigning this weighting as an impact value for the corresponding event, to allow an impact value to be assigned to an event where the user consumed food or drink.

In the above embodiment, a mathematical function or relationship is used to model each event. In one embodiment, the height of the first maxima after an event is taken as the weighting.

In a further embodiment, a computer implemented method for providing a user with an indication of the relative impact of food and drink on their body fluids is provided, comprising:
providing a user interface on a mobile device adapted to allow a mobile device to process user inputted event data of a plurality of events, wherein the events comprise the user consuming food or drink, the event data comprising the type of food or drink and the time at which said event occurred;
receiving data at the mobile device from a sensor, wherein the data received from the sensor comprises indicator information over the time corresponding to said plurality of events, said indicator information being a numerical measure of a physiological parameter;
transmitting said indicator information and event data to a server;
receiving data from said server comprising an impact value for the plurality of events with the corresponding events, wherein the impact value is an indication of the impact of the event on the body fluids of the user; and
displaying on said mobile device, food and drink ranked using said impact value.

In a yet further embodiment, a carrier medium carrying computer readable instructions adapted to cause a processor to perform the above method is provided

In a further embodiment, an apparatus for determining the relative impact of food and drink on the body fluids of a user is provided, the apparatus comprising a processor, said processor being adapted to:
receive event data of a plurality of events, wherein the events comprise the user consuming food or drink, the event data comprising the type of food or drink and the time at which said event occurred;
receive indicator information over the time corresponding to said plurality of events, said indicator information being a numerical measure of a physiological parameter;
fit a mixture of probability density functions to the indicator information, wherein each probability density function represents the probability that an event of the plurality of events impacted the indicator information at a time; and
extract a weighting from each fitted probability density function and assigning this weighting as an impact value for the corresponding event, to allow an impact value to be assigned to an event where the user consumed food or drink.

The sensor, may be configured to collect data concerning the body fluids of a user and output said data as indicator information,
the processor being adapted to receive said indicator information output from said sensor.

The apparatus may further comprise mobile device such as a mobile telephone, tablet, smart watch etc, said device being communicatively coupled to said sensor such that the output of the sensor is received by the mobile phone, the mobile phone being adapted to send data to a server housing the said processor, the server being adapted to send a plurality of impact values and corresponding events to the mobile telephone.

In a further embodiment, an apparatus for providing a user with an indication of the relative impact of food and drink on their body fluids, the apparatus being a mobile device comprising:
a user interface adapted to allow the user to input event data of a plurality of events, wherein the events comprise the user consuming food or drink, the event data comprising the type of food or drink and the time at which said event occurred;
a first receiver adapted to receive data from a sensor, wherein the data received from the sensor comprises indicator information over the time corresponding to said plurality of events, said indicator information being a numerical measure of a physiological parameter; and
a transmitter configured to send said indicator information and event data to a server;
a second receiver adapted to receive data from said server comprising an impact value for the plurality of events with the corresponding events, wherein the impact value is an indication of the impact of the event on the body fluids of the user,
the mobile device comprising a processor, said processor being adapted to display on said mobile device, food and drink ranked using said impact value.

Methods in accordance with embodiments of the present invention can be implemented either in hardware or on software in a general purpose computer. Further methods in accordance with embodiments of the present can be implemented in a combination of hardware and software. Methods in accordance with embodiments of the present invention can also be implemented by a single processing apparatus or a distributed network of processing apparatuses.

Since some methods in accordance with embodiments can be implemented by software, some embodiments encompass computer code provided to a general purpose computer on any suitable carrier medium. The carrier medium can comprise any storage medium such as a floppy disk, a CD ROM, a magnetic device or a programmable memory device, or any transient medium such as any signal e.g. an electrical, optical or microwave signal.

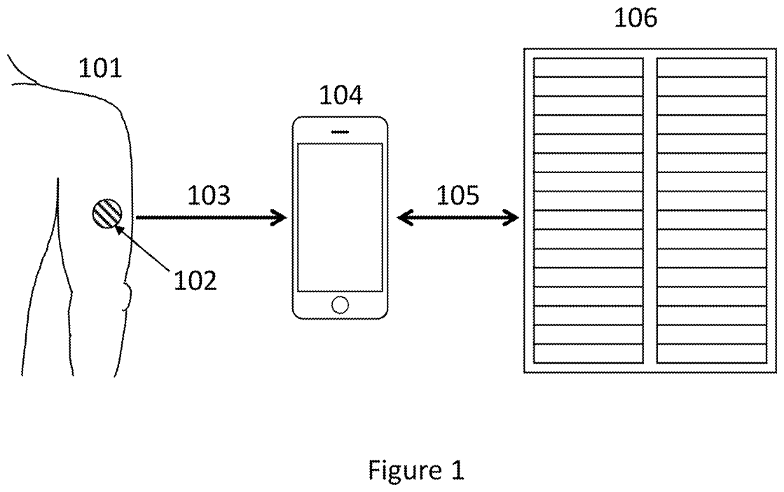

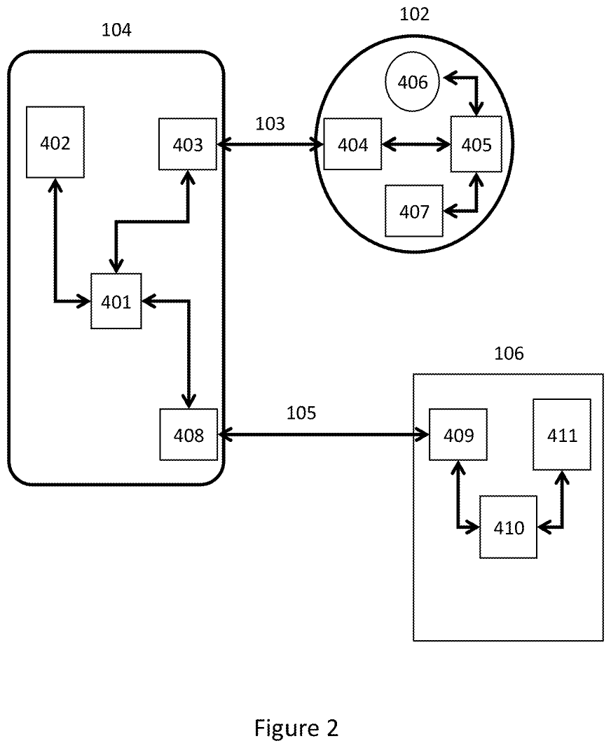

FIGS. 1 and 2 is a schematic of the apparatus. In an embodiment, a sensor 102 which may be provided on the arm of a user 101. The sensor serves to measure information concerning a physiological parameter of the user, such as the body fluids of a user (referred to an “indicator information”), for example, information concerning solutes in the body fluids, such as blood or cellular interstitial fluid. The solutes being selected from glucose and other blood sugars, or, ketones, insulin, cortisol, oxygen, grehlin and other hormones.

The sensor 102, sends via wireless signal 103 this data to mobile device 104. The mobile device 104 provides a user interface to allow the user to input data relating to events such as their food and/or drink intake, their activity level and other matters that might affect their body. Other examples could be stress levels etc.

The mobile device 104 then sends the data from the sensor and the data inputted by the user. The data from the sensor and the data from the user may be sent together or separately via signal 105 to a server 106. The server then processes the mix of information concerning the indicator information and the event information and returns to the mobile 104, information identifying the impact that identified food and drink items have on the user's body.

In this embodiment, the processing of the data largely takes place at the server, but in other embodiments, all processing of the data could take place at the mobile device.

The mobile device 104 is shown as a mobile telephone, but may be any type of mobile device, for example, a smart watch, tablet etc.

In more detail, the sensor is a continuous glucose monitor (CGM) 102 that is provided on the arm of a user. However, the CGM may be attached to any part of the body, for example the leg or the torso. The CGM has a needle which carries a tube on the needle's exterior surface. The CGM is applied to the user's skin, the needle and tube piercing the skin. The needle and tube are passed sufficiently far into the skin until one opening of the tube is within the interstitial fluid and the other opening is outside of the user's skin. The wall of the tube may be porous so as to more easily permit the movement of interstitial fluid through the tube. The CGM has an adhesive surface that will cause it to adhere to the user's skin when applied. The needle is then withdrawn from the skin, leaving the tube in place. This tube carries interstitial fluid from the user's arm into the CGM housing to contact a glucose concentration sensor 406.

The glucose concentration sensors of the type used in the CGM 102 of this embodiment use enzyme electrodes to measure glucose concentration. Enzyme electrodes use enzymes which catalyse reduction-oxidation (redox) reactions and in doing so accept or donate electrons. This movement of electrons may be exploited to produce a concentration-dependent current, voltage, or resistance which may be measured using electrodes. Glucose oxidase produces hydrogen peroxide which is directly proportional to the concentration of glucose. Peroxide may be measured amperometrically using a platinum electrode. Glucose oxidase is specific for glucose but is able to use alternative oxidising sources to molecular oxygen. Using alternative electron donors in the reaction allows it to be independent of local oxygen concentrations and these alternatives are known as mediators. These mediators can then be measured amperometrically to determine glucose concentration.

It should be noted that any technique for measuring blood glucose concentration may be used. Examples of these measuring techniques include Subcutaneous amperometric electrodes (i.e. the technique described above), microdialysis, intravenous implantable devices, micropores/microneedles, reverse iontophoresis, sonophoresis, fluorescence, infrared spectroscopy, Raman spectroscopy. It should also be noted that other sensing devices for measuring blood glucose concentration may be used including but not limited to wearable electronic devices, such as smart watches, smart rings and fitness trackers, which have the capability to perform any technique for measuring blood glucose concentration measurement.

The concentration of solutes such as that of glucose in the interstitial fluid reflects that of the concentrations in the blood. By sampling the concentration of glucose in the interstitial fluid, this provides an accurate approximation of the concentration of glucose in the blood without the need to extract blood or otherwise find and penetrate a blood vessel.

The glucose concentration sensor 406 measures a current, voltage or resistance, the value of which is sent to the CGM processor 405. The processor 405 converts this current into a glucose concentration value which is then stored in the CGM memory 407 in conjunction with a value for the time at which that recording was made. The processor 405 records values from the glucose concentration sensor 406 at regular intervals. CGMs of this form may have a limited memory which does not permit the storage of large amounts of data. As such, if the memory 407 of the CGM 102 is full, the processor 405 can overwrite the oldest value in memory with the most recent value and/or overwrite selected older values such that older data is saved at a lower frequency. This creates a rolling buffer of data that continuously replaces older data with newer data as necessary.

In an embodiment, the sensor will perform sample readings once per minute. However, in order to save memory space, the once samples are over 16 minutes old, their data is overwritten such that is it retained at a lower sampling frequency, for example, after the most recent 16 minutes, only samples taken every 15 minutes are maintained. Samples older than 8 hours 16 minutes are then discarded.

The above has explained that in embodiments, sensors and other sensing devices can be used that penetrate the skin or those that do not

Further, the above discusses continuous measurements. For the avoidance of doubt, it should be noted that continuous refers to the operation of the sensor and not necessarily the form of the output data. For example a continuous sensor may be capable of outputting either a single unbroken measurement over time or a plurality of discrete measurements.

The above has also mainly referred to a glucose sensor. However, such sensors often measure ketones or other blood solutes.

A mobile device 104 of the user 101 is communicatively coupled with the CGM 102. This connection is made using a Near-Field-Communications (NFC) antenna 403 on the mobile device and a corresponding NFC antenna 404 on the CGM 102. However, it is to be understood that any connection method such as Bluetooth, Wi-Fi, 2.4 GHz radio transmission, other wireless transmission, or wired connection could be used to make a connection 103 between the mobile phone 104 and the CGM 102. These connections can be short-range and so the user may be required to bring the mobile device 104 into close proximity with the CGM 102 in order for a connection to be established. This connection may be triggered automatically in response to the mobile device NFC antenna 403 being brought within range of the CGM NFC antenna 404.

In an embodiment, the mobile device NFC antenna 403 sends a signal to the CGM NFC antenna 404 to request that the data stored in the CGM memory 407 be sent to the mobile device 104. This signal is received by the CGM NFC antenna which is then passed to the CGM processor 405. The CGM processor 405 retrieves the data stored in the CGM memory 407 and sends it to the CGM NFC antenna 404 to be transmitted. The CGM NFC antenna 404 transmits the data 103, which is received by the mobile device NFC antenna 403. The data is then passed to the mobile device processor 401 which then stores the data in the mobile device memory 402.

This data received from the CGM 102 is in a binary string format to minimise data storage usage in the CGM memory 407. The data is taken from the mobile device memory 402 by the mobile device processor 401 and converted into a format which may be more readily processed. This could be in a dictionary collection format with recordal timestamp values paired with glucose concentration values. The data after processing is then recorded in the mobile device memory 402.

In an embodiment, the mobile device 104 is used to scan the sensor to retrieve data from the sensor. However, in other embodiments, the sensor may be configured to push data to the mobile device.

The data stored in the mobile device memory 402 is retrieved by the mobile device processor 401 and is sent to the mobile device network adapter 408. The network adaptor 408 sends the data 105 to a server 106 via the server network adapter 409. The connection between the mobile device 104 and the server 106 is via the internet (not shown). Other connection methods between the mobile device such as via transfer by a data storage medium (e.g. a memory card) can be used. This data received at the server network adapter 409 is then passed to the server processor 410 which then records the data in the server memory 411.

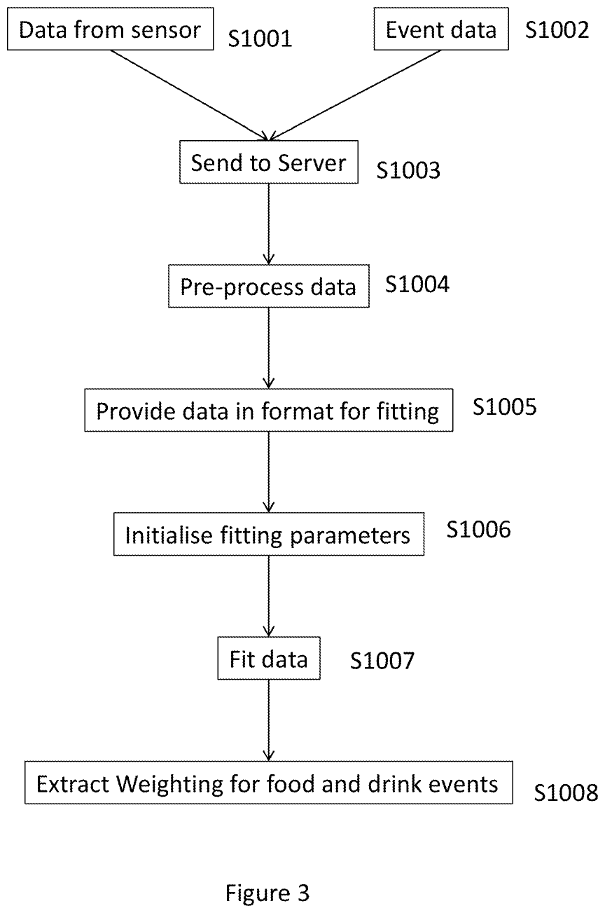

There are various options for the arrangements of mobile device and server which may communicate with the sensor to perform the method which will be described next with reference to FIG. 3. The mobile device 104 has a processor which will execute a program or “app” for receiving the data collected by the sensor 102 and processing the data either by itself or by communicating with the server 106. A single app may be provided on the mobile device 104 or there may be multiple apps involved to perform the method which will be described with reference to FIG. 3. For example, a sensor app may be provided specifically for communicating with the sensor 102 and this sensor app may be configured to receive the data from the sensor 104 and then pass to another app for the rest of the processing that will be described with reference to FIG. 3. The sensor app may provide its data directly to the other app or may communicate with the other app via a server.

The sensor app may just export raw sensor data or may provide some further pre-processing of the data. The sensor app may be configured to be health or fitness app which receives data from many different sensors. The sensor app may also receive the data from the sensor via a server.

While the above describes an embodiment where the data received by the mobile device 104 is sent directly to the server 106, in a further embodiment a facilitating server may be used, not shown in FIG. 2. The facilitating server may be provided between the mobile device 104 and the server 106, such that the retrieved data is first sent from the mobile device 104 to the facilitating server. The facilitating server may then forward the data to server 106. While the mobile device 104 may communicate indirectly with the server 106 through the facilitating server, it is possible that the server device 106 may return information identifying the impact that identified food and drink items have on the user's body directly to the mobile device 104, without sending the information to the facilitating server first. It is also possible however, that the server 106 first sends the information identifying the impact that identified food and drink items have on the user's body to the facilitating server, which then forwards the information to the mobile device 104.

In a further embodiment, the sensed data may be communicated directly from the sensing device 102, such as a wearable electronic device, to the server 106 or the facilitating server. In turn, the facilitating server forwards the sensed data to the server, where the processing of the data is performed. The server 106 may then return the information identifying the impact that identified food and drink items have on the user's body to the mobile device 106 directly or through the facilitating server, as explained above.

FIG. 3 is a flow chart demonstrating the basic processes. In an embodiment, these processes are split between a mobile telephone and a server. However, all processes may be performed on a mobile telephone.

In step S1001, data from the sensor is obtained by the mobile telephone. The details of this mechanism have been previously described with reference to FIG. 2.

The sensor data is processed by the mobile phone 104 such that it can be read in the form of a glucose measurement and the timestamp at which that measurement was taken. In addition to the glucose measurement of the timestamp, the mobile phone 104 also sends data indicating the sensor to the server and also, potentially data indicating the age of the sensor. The age of the sensor 102 is used in this embodiment as the sensor will be calibrating itself during a predetermined time at the start of its life, for example, one hour. Therefore, data recorded in this predetermined time is likely to be unreliable. Also, the sensors have a fixed lifetime, for example, 2 weeks. Therefore, the age of the sensor 102 will also indicate whether the sensor has expired.

In addition to the data from the sensor in step S1001, event data in step S1002 is also collected. In an embodiment, the event data is provided to a mobile phone via user input.

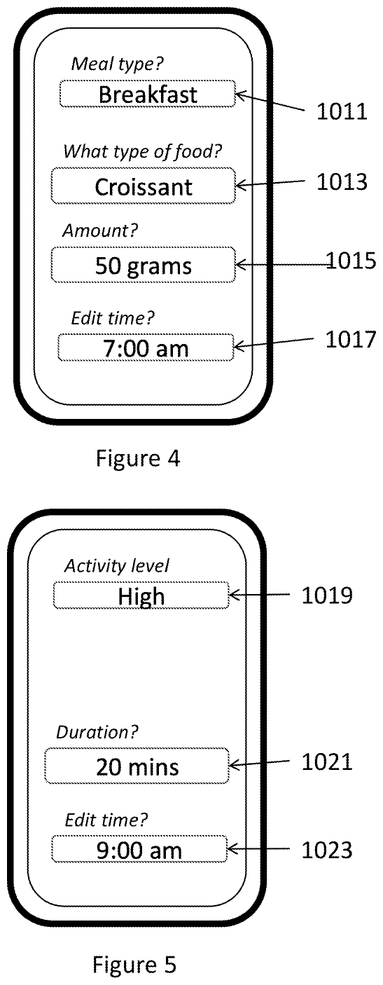

FIG. 4 shows a schematic of a possible data entry screen for the event data. In data entry field 1011, the mealtime is entered. This will typically be provided by a drop-down menu and in this example, the mealtime is shown as “breakfast”. However, in other embodiments, this could be a free text entry field for the user where the user could enter, for example “after-school snack”. In field 1013, the user will enter the type of food. The type of food can be entered by selecting an entry from a food database. For 50 example, the type of food or drink can be selected from a drop-down menu, listing some or all entries in the food database. Alternatively or additionally, the type of food or drink can be entered freely by the user. For example, a type of food can be freely entered by the user if the type of food is not saved in the database.

In field S1015, the amount of food consumed is added. Typically, the amount of food consumed by the user would be accurately indicated in grams. For example, as shown in the FIG. 4, if the user has consumed a croissant, which weights 50 grams, then the amount would be input as 50 grams. The amount of food can be indicated in other units of measurement. Furthermore, the amount of food may alternatively be added using a more rough indication, for example the user can indicate whether they have had one portion or other serving size of the type of food.

In an embodiment, the event will be time stamped at the time when the user enters the data. However, it is appreciated that sometimes the user might enter the data later in the day and therefore, field 1017 allows the user to edit the time of the event.

FIG. 4 shows the entry screen for food & drink. However, the user can also enter other event data not related to food & drink. In an embodiment, the method described herein will be able to compensate for other events that could affect the levels of the user.

FIG. 5 shows a typical entry screen for entering other event data. In entry field 1019, the activity level of the user is entered. In an embodiment, the screen will give the user some indication of what types of activity level constitute a high level or a low level.

In field 1021, the user will enter the duration of the activity for example if they ran for 20 minutes, then the duration would be 20 minutes. In field 1023, the user is asked if they wish to edit the time from the current time. It will be appreciated that in further embodiments, it is possible that the mobile phone may be able to establish data relating to activity itself or from another sensor on the body, for example a fit-bit or the like.

In an embodiment, the mobile phone will send data to the server in step S1003 once it has received either event data or data from the sensor. The data from the sensor will be in eight-hour chunks. However, it should be noted that the eight-hour chunks of data will be stitched together at the server as this data is time stamped. Due to the different sampling rates saved by the sensor, where possible, where two records overlap, the record with a finer grained data will be used.

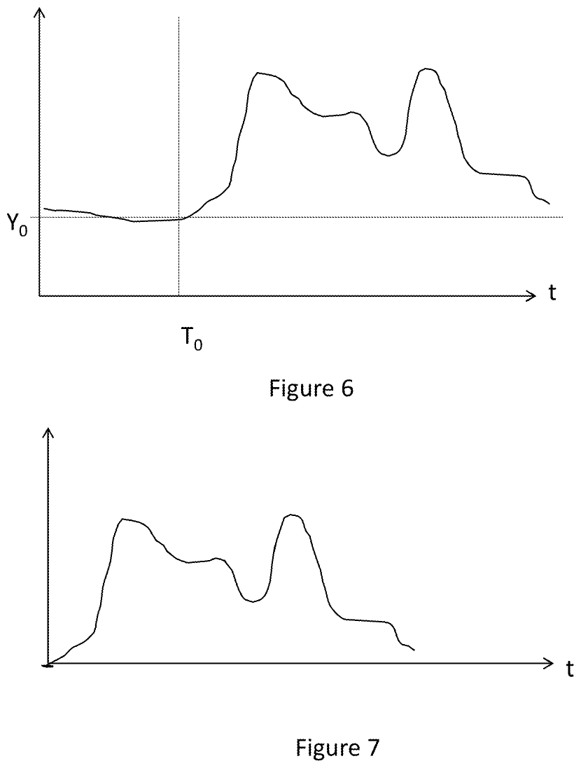

In step S1004, the data is initially pre-processed. FIG. 6 is a rough schematic of typical data. At the start, the server will identify what will be termed time T0. Time T0 is a point at the end of four or more hours of inactivity. The period of inactivity will be determined from the event data.

Next, the data after T0 will be considered (the time and the data before time T0 will be thrown away). This data will be referred to as data in the analysis period. In an embodiment a base value Y0 for this data will be identified. Next, Y0 will be subtracted from all of the data to produce the pre-processed data in FIG. 7.

In an embodiment, as described above, Y0 is the minimum value of the data in the analysis period. However, other methods may be used for determining a base value Y0, for example,
1. The last glucose reading before the event time (food/drink) if there were no other meals in a predetermined time period (referred to as a “period of inactivity”) preceding the meal of interest, or
2. The first glucose reading before the period of inactivity. Thus, for example, if several foods were consumed within less than 2.5 hours of each other the glucose reading that precedes all of these foods would be taken.

The period of inactivity could be 2.5 hours, 4 hours etc.

In an embodiment, which of the above approaches is used might depend on the user. For example if the person's starting glucose levels is very high and then drops throughout the day—a lot of zeros (negative values are clipped to zero) if a value from the start of the session is used as the activity level.

Also if the user's data shows a trend unrelated to food (e.g. if it drops throughout the day for a particular use) then this background could be subtracted instead.

Further examples of determining a base value Y0 could be to assume a base value for the whole day to be the level after waking up (or some average of all readings after waking up but before consuming any foods for that day).

Also, the inactivity period could be increased or decreased from 2.5 hours. The inactivity period might also be food specific (some meals take longer to digest and some users may be more insulin resistant than others so the glucose response for a meal may take a longer time to complete). Also, in an embodiment the last glucose reading before the meal if there was a period of inactivity is used, but it might be possible to take a reading that's say 15-30 minutes before the meal time, especially for users that do not log their meal times very accurately.

In an example, users who suffer with diabetes will have a different glucose response and this can be considered while setting Y0.

In an embodiment, a base value may be based on a daily trend rather than a single value.

For example, periods of inactivity longer than 4 hours will be determined from the event data, and the food events in between two subsequent periods of inactivity will be combined in a group together. The base value for a group of events will then be determined based on the reading preceding the first food event in the group. If the impact levels in the event data in said group drops below 95% of the initial base value of the group then the group is split up into further groups. The base value for each of the further groups will be determined based on the reading preceding the first event in the further group. The further groups may be further split up into further groups, following the same procedure.

Next, in step S1005, the pre-processed data from step S1004 will be converted into a format for fitting. The data will be fitted using probability distribution functions (pdf). Specifically, it is assumed that each event can be modelled by a corresponding probability distribution function.

In step S1005, the data is converted to a format suitable for fitting. In this embodiment, this is done by taking each pre-processed glucose level where the number of data points is the glucose value and the values that they take is the time stamp of the event in seconds. So for example: a pre-processed glucose value of 2.3 at an event time of 2 minutes would be transformed to 23 instances of 120 seconds.

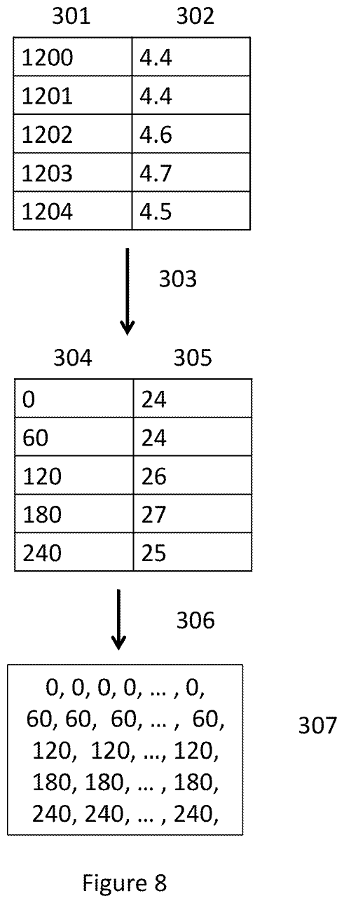

FIG. 8 is a summary showing the data processing steps according an embodiment. The upper most panel of FIG. 8 shows the data of the type used to produce the raw data of FIG. 6. The time stamp data is shown in column 301 and the corresponding sensor reading in column 302.

This is then converted in process 303 so that the time data is converted to the time data in column 304 where T0 has been subtracted. The time data is also shown in seconds. The corresponding indicator information is shown in column 305. For this data set, a base value (Y0) of 2 is used. The indicator information has this base value subtracted and has been multiplied by 10 to produce integer readings.

This is then converted in step 306 to the data shown in table 307 where the number of instances of each time stamp is repeated for the integer number of glucose readings to which it corresponds.

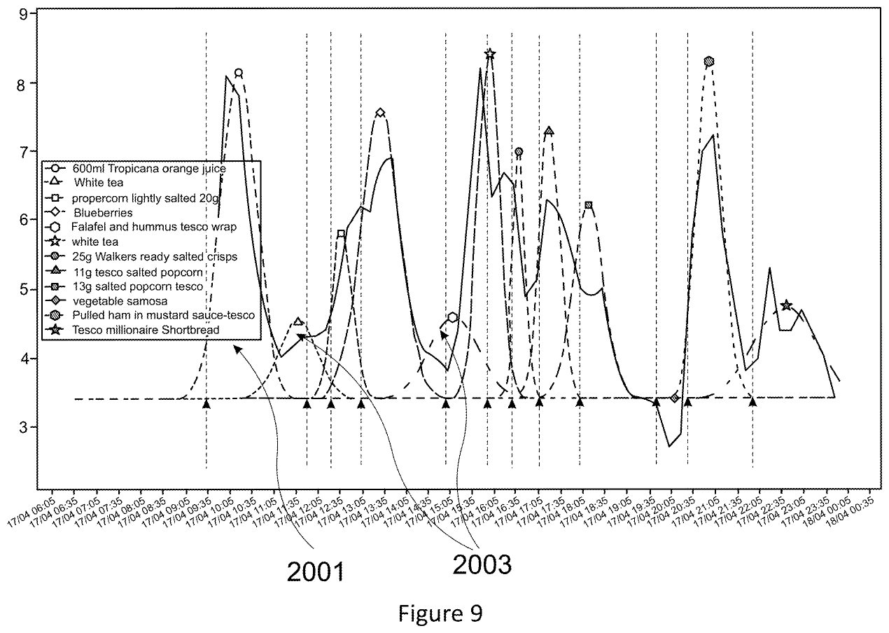

By fitting the events each with its own probability density function, a pdf mixture model is produced for the plurality of events. In an embodiment, the pdfs are Gaussian distributions as shown with reference to FIG. 9. In FIG. 9, the solid line is the actual data, the dotted curves show the fit of the normal pdf distributions. The events are shown as vertical dotted lines and the foods and drink consumed for each event are shown in the key. In the fit, it is assumed that each event corresponds to a single distribution.

However, although there is a good fit to the peaks as shown, for example by feature 2001, some of the peaks are not so well fitted as shown by the attempts to fit the distributions shown by features 2003.

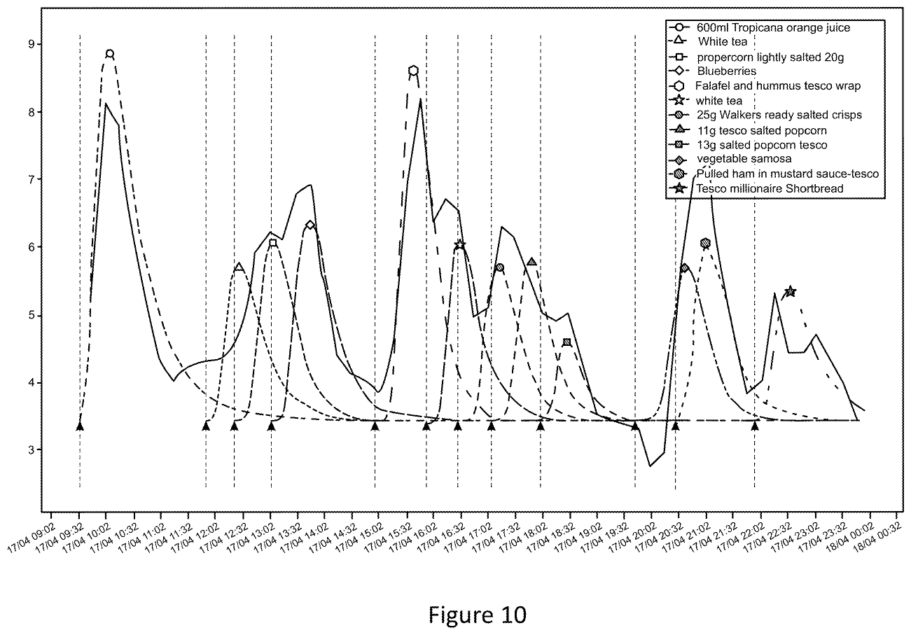

Therefore, in a further embodiment, a non-symmetric pdf is used. Here, the asymmetric pdf is the so-called lognormal pdf. The results of fitting the same data as shown in FIG. 9 with a lognormal pdf are shown in FIG. 10.

Here, the asymmetric lognormal pdf f(x) can be modelled as:

$$f ( x ) = \frac{1}{x σ \sqrt{2 π}} {e}^{- \frac{{( ln x - μ )}^{2}}{2 {σ}^{2}}}$$

Where x is the variable (in this case time), μ is the mean of the variable's natural logarithm and σ is the standard deviation of the variable's natural logarithm.

In addition to fitting μ and σ, two other variables are fitted, these are T and π. Where T is the timeshift of the probability distribution from the origin, and the origin being at x=T₀.

This can be thought of as writing x′=x−T and where the probability distribution is determined for x′. πᵢ is the weighting applied to each probability distribution i and there are N distributions to be fitted where N corresponds to the number of events identified from the event data.

In this embodiment, the following formula is fitted:

$$P ( x ) = {\sum }_{i = 1}^{N} {\prod }_{i} {f}_{i} ( x )$$

where:

$${f}_{i} ( x ) = \{ \begin{matrix}\frac{1}{( x - {T}_{i} ) {σ}_{i} \sqrt{2 π}} & {e}^{- \frac{{( ln ( x - {T}_{i} ) - {μ}_{i} )}^{2}}{2 {σ}^{2}}} \\ 0 & x \leq {T}_{i}\end{matrix}$$

and the impact value is derived from:

impact valueᵢ=πᵢ*Peak Height of fᵢ(x)*converting parameter

Where Tᵢ are the times of the i^{th} food (minus the time of the first food in that block represented by T₀).where the peak height of f(x) is determined using:

$$Peak Height of {f}_{i} ( x ) = \frac{1}{\sqrt{2 {πσ}^{2}}} {e}^{- μ + {σ}^{2} / 2}$$

and the converting parameter is determined using:

$$converting parameter = \frac{total length of data in format for fitting * 60}{10}$$

where the data in format for fitting is the data in table 307, for example, 10 and 60 are corresponding to the values used in step 304 to convert the pre-processed glucose values from one decimal values to integers and event time in minutes to event time in seconds, the two values can be considered more generally to be parameters relating to the “number of decimal places” and the “time multiplier”. Of course, these parameters can be adapted as necessary. Thus, the converting parameter is used to convert the probability distribution values back to sugar level values, such that the impact value i, also referred to as the impact score, of each event is presented as a sugar level value.

In an alternative embodiment, it is possible to use the area under the curve (AUC) of P(x), instead of the peak height of P(x) to determine the impact value, using:

impact valueᵢ=πᵢ* AUC_{Total}*converting parameter

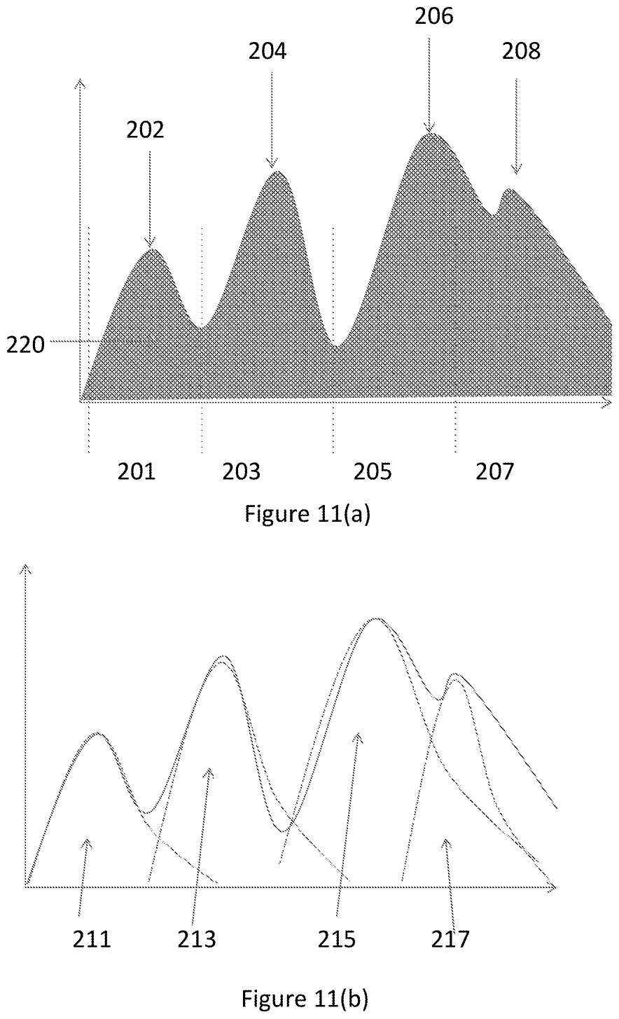

How this is done will be described with reference to FIG. 11(a).

FIG. 11(a) shows a schematic of data to be fitted after pre-processing. Four events have been identified in the event data 201, 203, 205 ad 207 each giving rise to a subsequent peak 202, 204, 206 and 208 respectively. The area under the graph AUC will be used to allow data from different activity periods to be directly compared. To fit data for an event, in an embodiment, at least 2 hours of data must be available after the event.

Referring back to FIG. 3, in order to fit the parameters T, πᵢ, μ and σ, the parameters are first initialised in step S1006.

In an embodiment, μ and σ are initialised by setting the pdfs so that the following condition is satisfied:
1) only 1.5% of the impact is present after 2 hours after the meal (i.e. the cumulative distribution function (7200 secs)=0.985)
2) half of impact is before 50 minutes/half after (i.e. median is at 3000 seconds)

Other ways of initialising u and o are also possible. For example, for some users, such as diabetic or pre-diabetic users, more than 1.5% of the impact is present after 2 hours after the meal. Therefore, it is also possible that the μ and σ are initialised based on the user's individual data. It is also possible to initialise μ and σ based on the macronutrients of the type of food, as defined in the food database Pᵢ, for example.

T is initialised by setting T to the time of the event to be fitted. πᵢ can be initialised in a number of different ways. In one embodiment, πᵢ is initialised by being set to a value that is the reciprocal of the number of events. For example, if there are five events to be fitted, then πᵢ is initialised to ⅕ for each event.

However, in other embodiments, the value of πᵢ can be taken from a database based on the description of the event to which it relates. For example, if the event relates to food or drink, a previously stored value of πᵢ for that food or drink could be used. This previously stored value could relate to the average value stored for all users of the system, subsets of users who have or are expected to produce similar responses, the average value for the food/drink of the particular user or any combination thereof.

In step S1007, the data is then fitted using the above mixture model and expectation maximisation (EM). FIG. 11(b) shows an example of the fitting where there are 4 events and 4 probability distributions 211, 213, 215 and 217 are fitted to the events. However, other probabilistic inference techniques could be used, for example based on Bayesian methods. It is possible for the data to be fitted using a mix of distributions, for example both Gaussians and lognormal distributions.

In step S1008, the weighting πᵢ is then extracted for food and drink events. In order to allow the impact value to be used to compare data from different time periods, for example, the time period before the identified 4 hours with an event, the weighting πᵢ is multiplied by the height of the peak and the converting parameter and sent back to the mobile telephone 104. Alternatively, the weighting πᵢ may be multiplied by the area under the curve (AUC), for example, if the area under the curve is used in the impact values estimation, as explained above. This impact value is then used to rank the various food and drink events.

This will give the user the information they need concerning how various food and drinks affect their glucose levels and will allow them to select a particular food type which will minimise their risk of a glucose spike and potentially developing diabetes type II in future. As increase in the glucose levels, i.e. a glucose spike, causes an increase in the insulin levels in the body of a person, exposure to high levels of insulin, can cause insulin resistance to develop in some people, which in turn can lead to Type II Diabetes. It has also been observed that the heights of glucose spikes, and subsequent crashes is directly correlated to feelings of hunger in a person during the day. Therefore, a user can use the impact value to determine food and drinks which cause high glucose spikes and thus avoid them or reduce their consumption, and thus reduce their risk of developing diabetes. Users may also use the impact value to plan their meals during the day in order to ensure they intake the right amount of food without feeling hungry during the day and in-between meals. A weight decay variable may also be integrated to deal with the long tails of the lognormal distributions and therefore prevent glucose impact at time t being incorrectly attributed to food and drink events which were consumed many hours before it. Furthermore, integrating weight decay variable in the estimation of the sugar level impact values of food and drinks also eliminates the discrepancies between the glucose peaks and corresponding food and drink events that may arise when the user has forgotten to log the food or drink event.

In an embodiment, the weight decay variable is given by exponential distribution gᵢ(x), where

gᵢ(x)=λe^{-λ(x-(T}^{ᵢ}^{+D))}

and the weight decay variable is integrated into the mixture module by multiplying each distribution value by an exponential distribution, and the formula fitted can be rewritten:

$${P}^{′} ( x ) = {\sum }_{i = 1}^{N} {\prod }_{i} {f}_{i} ( x ) {g}_{i} ( x )$$

where the overall mixture model with weight decay is denoted by P′(x), Tᵢ denotes the time at which the food was eaten i.e. the food event was logged by the user, D denotes the time after the food has been logged by the user and the time delay after which the exponential weight decay comes into effect, and λ is the rate parameter of the exponential distribution.

In an embodiment, the time delay D after which the exponential weight decay comes into effect is two hours and λ is chosen such that the half-life of the exponential distribution gᵢ(x) is equal to one hour.

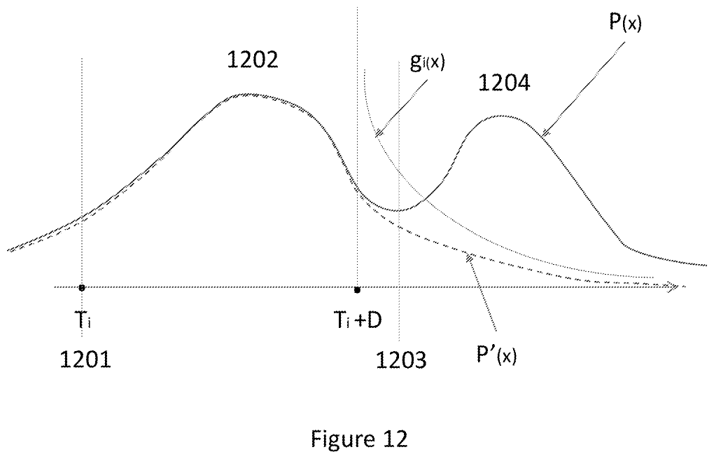

Furthermore, including the weight decay variable in the estimation of the sugar level impact also eliminates the discrepancies between the glucose spikes and corresponding food and drink events that may arise when the user has forgotten to log the food event. This is graphically illustrated in FIG. 12.

FIG. 12 shows a plot of the sugar level impact caused by two events, first event 1201 and second event 1203. The first event 1201 and the second event 1203 each give rise to a peak 1202 and peak 1204, respectively. Events 1201 and 1203 differ from each other in that the second event 1203 has not been logged by the user. In the Figure, P(x), denoted with a solid line, represents the overall sugar impact that is seen from the data while P′(x), denoted with a dashed line, represents the sugar level impact caused by logged events.

In an embodiment, the time after which the exponential weight decay comes into effect D and the parameter λ are fixed and they are not learned at the same time as the main process. However, it is also possible that the time after which the exponential weight decay comes into effect D and the parameter λ are learned separately from the main process and adjusted on per user and/or per type of food basis, for example.

In an embodiment, the time after which the exponential weight decay comes into effect D and the parameter λ are adjusted for each user by running the sugar impact scoring process, without making use of the weight decay variable, and by taking average values for μ and σ for the specific user.

Since o relates to how quickly the lognormal distribution decays, parameter λ is adjusted based on the average σ of the lognormal distributions of a specific person in relation to the overall average σ of the lognormal distributions across all users. For example, if a specific user has average σ higher than the overall average o across all users, the half-life of the user's widths decay exponential distribution can be increased, and thus the λ parameter is decreased.

The time after which the exponential weight decay comes into effect D is adjusted based on the average value for μ for the specific user, since e^{μ} gives the median of the lognormal distribution. Therefore, users with higher than the overall average μ would have higher average D than the overall average D across all users.

In an embodiment, the time after which the exponential weight decay comes into effect D and the parameter λ are adjusted on food by food basis, based on the average u and o for each type of food and drink.

The above describes one embodiment of fitting the event data. In a further embodiment, artefacts of the data derived either pre-or post pre-processing are identified. For example, such artefacts could be minimum, maximum and/or points of inflection.

In an embodiment, it is assumed that the next maximum or point of inflection after an event is linked to that event. In this situation, the height (Y value) of that maximum or point of inflection is used as the weighting and hence extracted as the impact value.

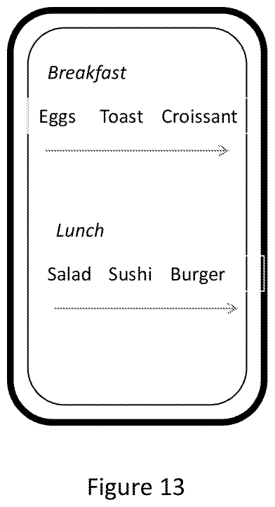

FIG. 13 shows a possible display. In the top part of the display, the heading “breakfast” is shown. Below breakfast, the 3 most popular food types that the user typically inputs as their breakfast show in order of increasing probability of causing a rise in glucose levels. This ranking of the 3 foods is achieved via the use of the impact value which has been calculated. In the bottom part of the display, similar data is shown for lunch.

In this example, the foods for each particular meal are ranked. However, it is also possible for the mobile to display all food/drink ranked in order of the probability of causing a rise in glucose levels. As this ranking of the foods has been derived from data of the user's actual changes in glucose levels in response to these foods, the ranking is tailored to the specific user. In addition, to ranking the foods for each particular meal, the food scores i.e. the impact levels, can be used by the users for comparison with other users, thus users can see how they compare to other users on the same food or drinks. The impact levels may also be used to group users who ‘spike’ from the same type of food or drinks.

Furthermore, the various types of food and drink can be ranked in different categories based on the impact values or food score for each type of food and drink. For example, foods with impact values under 1, can be classed in “spike free foods” category, type of food and drink with score between 1 and 2.5 can be ranked in a “medium spike foods” category, and types of food and drink with impact level over 2.5 are ranked in “high spike foods” category. The categories may be used to suggest to the user type of foods and drinks for various meal types i.e. breakfast, lunch or dinner, which will not cause a high glucose spike.

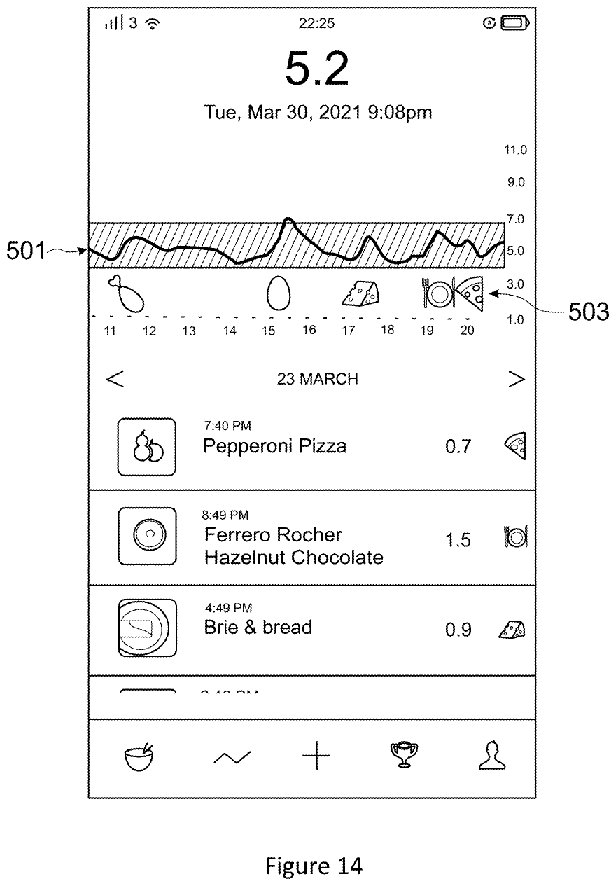

FIG. 14 shows a further possible display. The top of the display 501 shows a plot of the impact information over time. Below the plot, icons are shown which correspond to the food and drink 503 which correspond to the peaks shown in the impact information.

In the lower part of the screen, more information is given about each of the foods which are shown the cause the peaks in the impact information. The food is listed with its impact value and icon, the impact value is shown in a colour to quickly show to the user if it is a ranked low (green), medium (amber) or high (red). In FIG. 14, the impact values for pepperoni Pizza and Brie and Bread are less than 1 and so these values are displayed in green. The impact value for Ferrero Rocher is 1.5 and shown in amber.

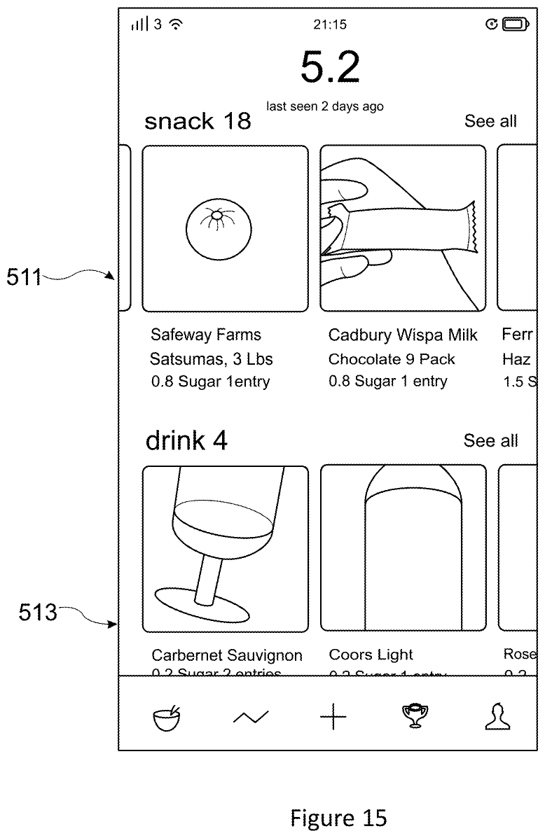

Possible foods and drinks can be suggested in categories. FIG. 15 is a display showing how food and drink options can be provided by the method. In the top part of the screen 511, the user is shown possible snacks and their impact values. Low impact values are shown in green. In the lower part of the screen 513, possible drinks and their impact values are shown. Just snacks and drinks are shown, but other categories such as “lunch”, “dinner” etc can be shown. In an embodiment, these categories are determined from the time when the user entered data logging the relevant event, for example when they ate the food. The user may also indicate categories for events and the user may also add new categories.

In an embodiment, the user data is collected over a predetermined time period, for example 2 weeks. This data is then continually used to advise the user. The user may periodically retrain the system, for example once every 3 months.

The above embodiment describes an unsupervised training system. There is no split of test and training data, it is all achieved in one process.

In some of the above embodiments, just P(X) is fitted to the indicator information. This may be done using Expectation-Maximisation (E-M).

In further, embodiments, the function P′(X) is fitted to the indicator information. In these embodiments, E-M may also be used. Here, the same process is followed for the Expectation Step for P′(X) as for the case without g(x) (i.e. P(X)), the responsibilities of each event are calculated for each data point These responsibilities basically represent the probability that the data point is caused by that event.

Decay weights for every combination of data points and events are then calculated. It will be 1 for any data point that is less than 2 hours later than an event, and will exponentially decay thereafter.

Each responsibility is then multiplied by its corresponding decay weight, and this combination is then used in the M Step instead of responsibilities, but otherwise the M Step is not modified.

The above method was followed as it enforces the prior belief (that impact of a food should exponentially decay after X hours) in a strict way, rather than allowing the algorithm to learn this which it might not naturally.

Thus, in embodiments where the weight decay variable is employed, on each iteration of the EM algorithm, the weights of each distribution are multiplied by the weight decay variable gᵢ(x). Once the distributions are learned, the final distributions values P(X) are multiplied by the weight decay variable gᵢ(x) to obtain the P′(X).

While certain embodiments have been described, these embodiments have been presented by way of example only, and are not intended to limit the scope of the inventions. Indeed the novel methods and systems described herein may be embodied in a variety of other forms; furthermore, various omissions, substitutions and changes in the form of methods and systems described herein may be made without departing from the scope of the inventions. The accompanying claims and their equivalents are intended to cover such forms of modifications as would fall within the scope of the inventions as defined by the appended claims.

1. A method of determining the relative impact of food and drink on the body fluids of a user, the method comprising:
receiving event data of a plurality of events, wherein the events comprise the user consuming food or drink, the event data comprising the type of food or drink and the time at which said event occurred;
receiving indicator information over the time corresponding to said plurality of events, said indicator information being a numerical measure of a physiological parameter;
fitting a mixture of probability density functions to the indicator information, wherein each probability density function represents the probability that an event of the plurality of events impacted the indicator information at a time; and
extracting a weighting from each fitted probability density function and assigning an impact value based on this weighting for the corresponding event, to allow an impact value to be assigned to an event where the user consumed food or drink.

2. A method according to claim 1, further comprising outputting a warning level for a food or drink, wherein said warning level indicates a rise in indicator information expected by consuming the food or drink, said warning level being determined from the impact value for said food or drink.

3. A method according to claim 2, wherein there are at least two warning levels, where one of the warning levels indicates food or drink for the user to avoid and the other one of the warning levels indicates food or drink for the user to consider.

4. A method according to claim 1, adapted to output a ranked list of food and drink using said impact value for ranking.

5. A method according to claim 1, further comprising retrieving from a database an impact value for a food or drink from a plurality of users, and providing to a user a comparison of the user's impact score for a food or drink with an average impact score from the same food of drink determined from the plurality of users.

6. A method according to claim 1, wherein the events further comprise the user changing a level of physical activity.

7. A method according to claim 1, adapted to determine the relative impact of food and drink on blood sugars of a user, wherein the indicator information is an indication of the blood sugar levels.

8. A method according to claim 7, adapted to determine the relative impact of food and drink on glucose levels of a user, wherein the indicator information is an indication of the glucose levels.

9. A method according to claim 1, wherein at least one of the probability density functions fitted to the indicator information is an asymmetric distribution.

10. A method according to claim 9, wherein the asymmetric distribution is a log normal distribution.

11. A method according to claim 1, wherein each probability density function of the mixture of probability density functions is a product of two distributions, wherein the first distribution is configured to model a peak in the indicator information and the second distribution is used to model the decay of the peak.

12. A method according to claim 11, wherein the second distribution is of the form of an exponential decay.

13. A method according to claims 11, wherein the second distribution comprises user dependent fitting parameters.

14. A method according to claim 13, wherein the user dependent fitting parameters are common to groups of users.

15. A method according to claim 11, wherein the first distribution is an asymmetric distribution.

16. A method according to claim 1, wherein fitting a mixture of probability density functions to the indicator information comprises initialising the probability density functions using the time of the event to be fitted and a value representing the weight of probability density function derived from the event data.

17. A computer implemented method for providing a user with an indication of the relative impact of food and drink on their body fluids, comprising:
providing a user interface on a mobile device adapted to allow a mobile device to process user inputted event data of a plurality of events, wherein the events comprise the user consuming food or drink, the event data comprising the type of food or drink and the time at which said event occurred;
receiving data at the mobile device from a sensor, wherein the data received from the sensor comprises indicator information over the time corresponding to said plurality of events, said indicator information being a numerical measure of a physiological parameter;
transmitting said indicator information and event data to a server;
receiving data from said server comprising an impact value for the plurality of events with the corresponding events, wherein the impact value is an indication of the impact of the event on the body fluids of the user, the impact value being determined by fitting a mixture of probability density functions to the indicator information, wherein each probability density function represents the probability that an event of the plurality of events impacted the indicator information at a time; and extracting a weighting from each fitted probability density function and assigning the impact value based on this weighting for the corresponding event, to allow an impact value to be assigned to an event where the user consumed food or drink; and
displaying on said mobile device, food and drink ranked using said impact value.

18. A non-transitory computer readable storage medium carrying computer readable instructions adapted to cause a processor to perform the method according to claim 1.

19. An apparatus for determining the relative impact of food and drink on the body fluids of a user, the apparatus comprising a processor, said processor being adapted to:
receive event data of a plurality of events, wherein the events comprise the user consuming food or drink, the event data comprising the type of food or drink and the time at which said event occurred;
receive indicator information over the time corresponding to said plurality of events, said indicator information being a numerical measure of a physiological parameter;
fit a mixture of probability density functions to the indicator information, wherein each probability density function represents the probability that an event of the plurality of events impacted the indicator information at a time; and
extract a weighting from each fitted probability density function and assigning an impact value based on said weighting for the corresponding event, to allow an impact value to be assigned to an event where the user consumed food or drink.

20. An apparatus according to claim 19, further comprising a sensor, said sensor being configured to collect data concerning the body fluids of a user and output said data as indicator information,
the processor being adapted to receive said indicator information output from said sensor.

21. An apparatus according to claim 20, further comprising a mobile device, said mobile device being communicatively coupled to said sensor such that the output of the sensor is received by the mobile device, the mobile device being adapted to send data to a server housing the said processor, the server being adapted to send a plurality of impact values and corresponding events to the mobile device.

22. An apparatus according to claim 20, wherein the sensor is adapted to perform a non-invasive measurement.

23. An apparatus for providing a user with an indication of the relative impact of food and drink on their body fluids, the apparatus being a mobile device comprising:
a user interface adapted to allow the user to input event data of a plurality of events, wherein the events comprise the user consuming food or drink, the event data comprising the type of food or drink and the time at which said event occurred;
a first receiver adapted to receive data from a sensor, wherein the data received from the sensor comprises indicator information over the time corresponding to said plurality of events, said indicator information being a numerical measure of a physiological parameter;
a transmitter configured to send said indicator information and event data to a server; and
a second receiver adapted to receive data from said server comprising an impact value for the plurality of events with the corresponding events, wherein the impact value is an indication of the impact of the event on the body fluids of the user, the impact value being determined by fitting a mixture of probability density functions to the indicator information, wherein each probability density function represents the probability that an event of the plurality of events impacted the indicator information at a time; and extracting a weighting from each fitted probability density function and assigning the impact value based on this weighting for the corresponding event, to allow an impact value to be assigned to an event where the user consumed food or drink,
the mobile device comprising a processor, said processor being adapted to display on said mobile device, food and drink ranked using said impact value.
